(19) United States
(12) Patent Application Publication
(10) Pub. No.: US 2024/0238581 A1
(43) Pub. Date: Jul. 18, 2024

(54) INTRAVASCULAR BLOOD PUMP OUTFLOW FLOW DISRUPTOR

(71) Applicant: Shifamed Holdings, LLC, Campbell, CA (US)

(72) Inventors: Ari RYAN, San Jose, CA (US); Daniel HILDEBRAND, Santa Cruz, CA (US); Daniel VARGHAI, Scotts Valley, CA (US); Adnan MERCHANT, Fremont, CA (US); Seyed Mostafa GHOREYSHI, Campbell, CA (US)

(21) Appl. No.: 18/559,231

(22) PCT Filed: May 6, 2022
(86) PCT No.: PCT/US2022/028029

§ 371 Date: Nov. 6, 2023

Related U.S. Application Data
(60) Provisional application No. 63/185,188, filed on May 6, 2021.

(51) Int. Cl. A61M 60/13; A61M 60/237; A61M 60/414

(52) U.S. Cl. CPC A61M 60/13; A61M 60/237; A61M 60/414

(57) ABSTRACT

Catheter blood pumps that include an expandable pump portion. The pump portions include a collapsible blood conduit that defines a blood lumen. The collapsible blood conduits include a collapsible scaffold adapted to provide radial support to the blood conduit. The pump portion also includes one or more impellers. The pump portion may also include a blood mover that is adapted to move blood to reduce stagnation, and is separate from the impeller.

### CROSS REFERENCE TO RELATED APPLICATIONS

This application claims the benefit of U.S. Provisional Application No. 63/185,188, filed May 6, 2021, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

This application incorporates by reference herein for all purposes the entire disclosures of the following applications: WO 2018/226991; WO2019/094963; WO 2019/152875; WO 2020 028537; WO 2020/073047; and WO 2020/247612.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally-invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously-inserted ventricular support device.

There is a need to provide additional improvements to the field of ventricular support devices and similar blood pumps for treating compromised cardiac blood flow.

### SUMMARY OF THE DISCLOSURE

The disclosure is related to intravascular blood pump and their methods of and manufacture.

A intravascular blood pump (e.g., 10) is provided, comprising a collapsible pump portion (e.g., 20), an impeller disposed within the collapsible pump portion near a pump outflow, and a rotatable blood mover (e.g., 164) axially spaced from the impeller and being disposed at least partially within the pump outflow.

In some embodiments, the rotatable blood mover is rotationally coupled to the impeller.

In other embodiments, the rotatable blood mover is adapted to be collapsible and expandable.

In some embodiments, the rotatable blood mover is not adapted to be collapsible and expandable.

In another embodiment, the rotatable blood mover is more rigid than the impeller.

In some examples, the rotatable blood mover comprises a plurality of radially extending fins (e.g., 166), and optionally includes a conical surface (e.g., 169) from which the plurality of fins extend.

In one embodiment, the rotatable blood mover has a radially outermost dimension less than a radially outermost dimension of the impeller when expanded, both relative to a long axis of a drive cable, optionally wherein the radially outermost dimension of the blood mover is less than half of the radially outermost dimension of the impeller when expanded.

In some examples, the rotatable blood mover is adapted to reduce at least one of blood recirculation or stagnation in the vicinity of the blood mover or the pump outflow.

In one embodiment, the impeller is fully disposed in the pump outflow.

In some embodiments, the pump outflow includes a radial flow component.

In other examples, the blood mover is not in contact with the impeller.

In some embodiments, the rotatable blood mover is rotationally coupled to the impeller with a drive shaft.

In other embodiments, the rotatable blood mover is part of a bearing housing that rotates when the drive shaft rotates.

A intravascular blood pump (e.g., 10) is also provided, comprising a collapsible pump portion (e.g., 20), an impeller disposed within the collapsible pump portion near a pump inflow, and a rotatable blood mover (e.g., 164) axially spaced from the impeller and being disposed at least partially within the pump inflow.

In some embodiments, the rotatable blood mover is rotationally coupled to the impeller.

In other embodiments, the rotatable blood mover is adapted to be collapsible and expandable.

In some embodiments, the rotatable blood mover is not adapted to be collapsible and expandable.

In another embodiment, the rotatable blood mover is more rigid than the impeller.

In some examples, the rotatable blood mover comprises a plurality of radially extending fins (e.g., 166), and optionally includes a conical surface (e.g., 169) from which the plurality of fins extend.

In one embodiment, the rotatable blood mover has a radially outermost dimension less than a radially outermost dimension of the impeller when expanded, both relative to a long axis of a drive cable, optionally wherein the radially outermost dimension of the blood mover is less than half of the radially outermost dimension of the impeller when expanded.

In some examples, the rotatable blood mover is adapted to reduce at least one of blood recirculation or stagnation in the vicinity of the blood mover or the pump inflow.

In one embodiment, the impeller is fully disposed in the pump inflow.

In some embodiments, the pump inflow includes a radial flow component.

In other examples, the blood mover is not in contact with the impeller.

In some embodiments, the rotatable blood mover is rotationally coupled to the impeller with a drive shaft.

In other embodiments, the rotatable blood mover is part of a bearing housing that rotates when the drive shaft rotates.

A method of operating an intravascular catheter blood pump is also provided, comprising expanding an expandable blood conduit (e.g., 30), expanding an expandable impeller (e.g., 42), rotating the impeller to move blood through the blood conduit towards an outflow, and moving blood in the outflow by moving a blood mover, wherein the blood mover is different than the impeller.

In some implementations, moving blood comprises rotating the blood mover.

In one example, moving blood comprises rotating a non-expandable and non-collapsible blood mover.

In other examples, expanding the expandable impeller causes a portion of the impeller to be within the pump outflow.

In some embodiments, moving blood in the outflow by moving the blood mover comprises one or more of reducing blood recirculation or stagnation in the vicinity of the blood mover.

In other embodiments, expanding the expandable impeller comprises expanding the impeller distal to the blood mover.

In one example, at no time is the blood mover expanded.

In some embodiments, expanding the expandable impeller comprises expanding the impeller such that it is or is not in contact with the blood mover.

In one example, rotating a drive mechanism (e.g., one or more of a drive cable or shaft) which causes the rotation of the impeller and the blood mover.

In some embodiments, rotating a drive mechanism (e.g., one or more of a drive cable or drive shaft) which causes the rotation of the impeller and the blood mover to rotate at different speeds.

A method of operating an intravascular catheter blood pump is also provided, comprising expanding an expandable blood conduit, expanding an expandable impeller, moving blood through an inflow of the blood conduit by rotating the impeller, and moving blood in the inflow by moving a blood mover, wherein the blood mover is different than the expandable impeller.

In some embodiments, wherein the blood mover is passive and configured to rotate as a result of blood flow generated by the expandable impeller. In another embodiment, the blood mover is not operatively coupled to a driveshaft of the expandable impeller.

### BRIEF DESCRIPTION OF THE DRAWINGS

FIG. 1 illustrates a portion of an exemplary intravascular blood pump.

FIG. 2 illustrates a portion of an exemplary intravascular blood pump.

FIG. 3 illustrates a portion of an exemplary intravascular blood pump that includes a blood mover separate from an impeller.

### DETAILED DESCRIPTION

Intravascular blood pumps may include a pump portion that includes a pump inflow and a pump outflow, a blood conduit and one or more impellers that move blood through the blood conduit. Exemplary blood pumps described herein can comprise an expandable blood conduit, a pump inflow, a pump outflow, and one or more expandable and collapsible impellers that are adapted to move blood through the blood conduit when activated.

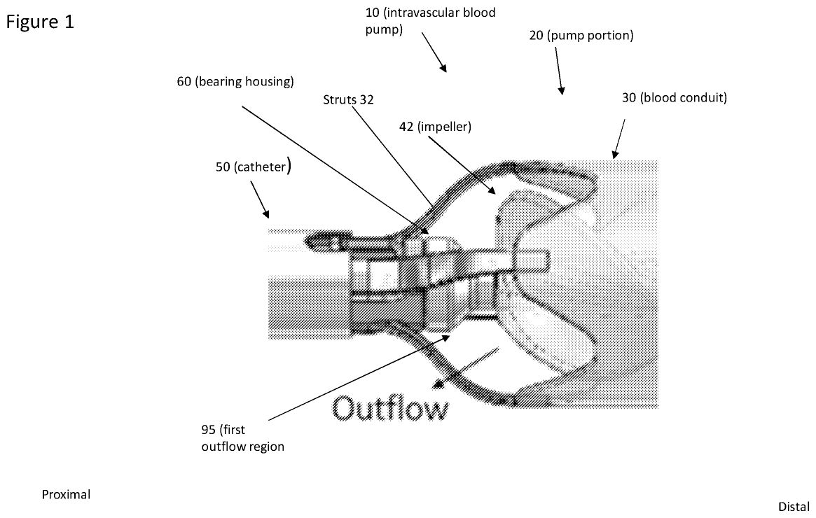

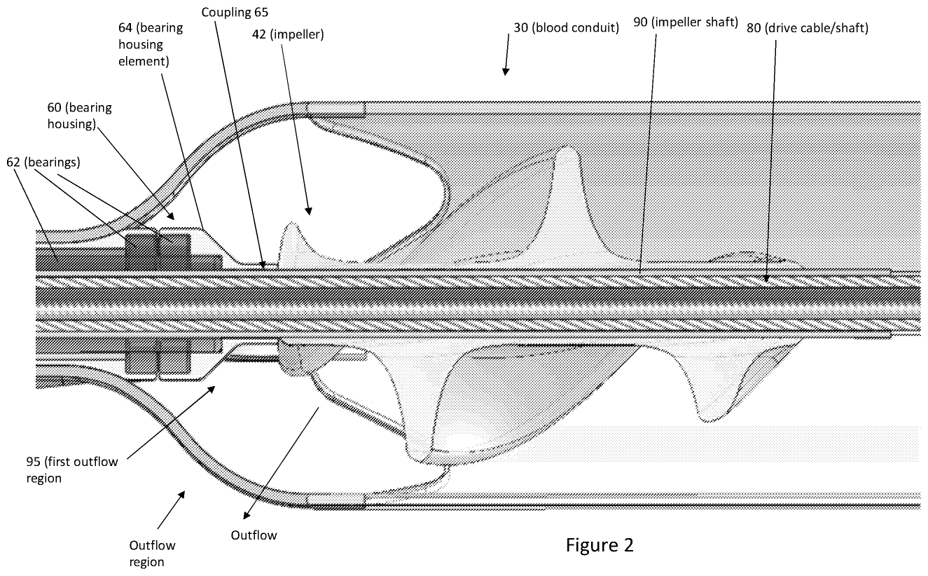

FIG. 1 illustrates a portion of an exemplary intravascular blood pump 10. Blood pump 10 includes a pump portion 20 that includes an expandable and collapsible blood conduit 30 (which may include a scaffold and one or more layers secured thereto) and struts 32 extending from the blood conduit 30 (struts on the other end not shown). Intravascular blood pump 10 also includes catheter 50 extending proximally relative to the pump portion 20, which may extend to an external motor. Blood pump 10 includes a rotatable drive mechanism (e.g., which may include one or more of a drive cable or drive shaft, such as drive cable 80 in FIG. 2) that is rotationally coupled to one or more impellers (e.g., impeller 42 shown in FIGS. 1 and 2), which may be rotated by a motor, which may be a motor external to the patient or which may be an onboard motor. Impeller 42 may be a proximal impeller such that the pump portion includes one or more impellers distal to the impeller 42.

As shown in FIGS. 1 and 2, in some embodiments a portion of the impeller may be disposed in the outflow. In this example, a proximal portion of impeller 42 is not completely surrounded by the blood conduit, as shown in FIGS. 1 and 2. This may at least partially contribute to the outflow having some radial blood flow component such that the outflow is not purely axial flow.

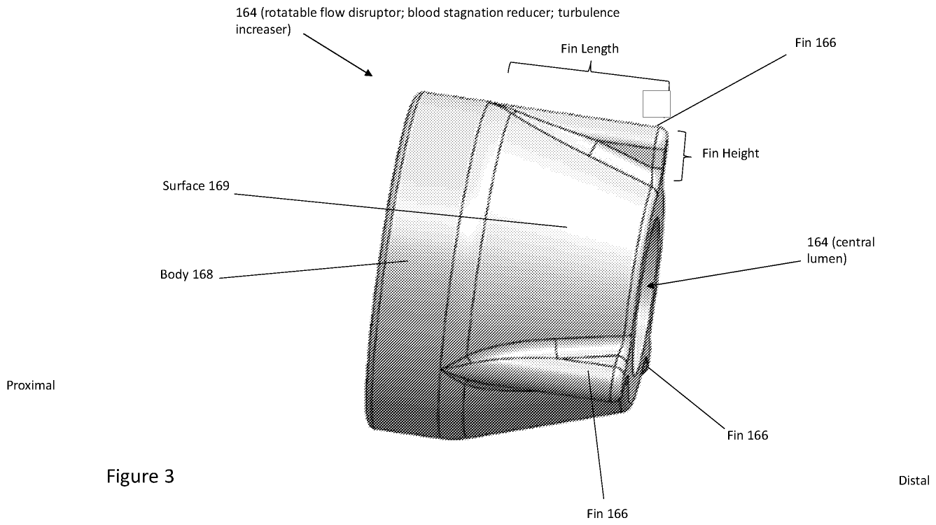

Blood pump 10 also includes bearing housing 60 (which may be a proximal bearing housing), which may house one or more bearings 62 therein, examples of which are shown in the sectional view of FIG. 2. In the exemplary embodiments in FIGS. 1-3, bearing housing 60 includes a bearing housing element 64 (or element 164 shown in FIG. 3), which in these examples is coupled to an impeller shaft 90 at coupling 65, and which rotates when the rotatable components rotate, both of which can be rotated by a rotatable drive mechanism. Housing element 64 may house a thrust bearing therein, for example. There may be some axial spacing or gap between bearing housing element 64 and the proximal end of impeller 42, or they may be in contact.

It is understood that bearing housing element 164 shown in FIG. 3 may be the housing element 64 from FIGS. 1 and 2, such that housing element 64 may be considered to be shown generally in FIGS. 1 and 2 without optional fins.

It is understood that element 64 or 164 in FIGS. 1-3 need not be part of a bearing housing, and it may still be adapted to perform any of the functions herein without being part of a bearing housing. For example, element 164 may be configured as a blood recirculation reducer and/or a stagnation reducer as those phrases are used herein without being part of a bearing housing. Element 164 shown in FIG. 3 (which may replace element 64 in FIGS. 1 and 2 and be disposed at the same location) is therefore also described herein as a blood recirculation reducer and/or a stagnation reducer, and may be configured as such.

Depending on the configuration of the outflow region of the pump portion of a particular intravascular blood pump, it may be beneficial for the blood pump to be adapted and configured to move blood within the outflow region. For example, it may be beneficial for the blood pump to include one or more components disposed at least partially within the outflow that are adapted and configured to move blood within the outflow region. As described herein, moving blood in this context may refer to one or more of reducing blood stagnation or reducing blood recirculation (optionally in the outflow or inflow region). There may be benefits to moving blood within the outflow of the blood pump or within the inflow. For example, there may be benefits to disrupting blood in the outflow region of the blood pump. For example only, moving blood within the outflow or inflow regions of the blood pump may reduce the likelihood of blood stagnating in a region of the outflow or inflow, such as at or near first outflow region 95 shown in FIGS. 1 and 2. This may (though not necessarily) be more likely to occur if the pump outflow includes a radial flow component, as opposed to purely axial flow at the outflow, for example. In the case of a radial or mixed flow impeller, for example, the flow has a radial component and thus there is less “washing” across the proximal bearing housing 60. Additionally, regardless of the radial and axial aspects of the blood flow at or in the vicinity of the outflow or inflow, there may be other advantages to having a blood mover in the inflow and/or the outflow. An exemplary benefit of reducing the likelihood of stagnating blood in one or more regions of the pump portions is to reduce the likelihood of blood clotting and the risk of thrombolic events associated with the pump.

FIG. 3 illustrates a merely exemplary blood mover 164, which as set forth herein may also be referred to as a blood flow disruptor, a blood recirculation reducer, and/or a blood stagnation reducer (or other derivative thereof). In the exemplary embodiments herein, blood mover 164 (which may take the place of element 64 in FIGS. 1 and 2) is adapted to rotate, and is rotationally coupled to impeller shaft 90, drive cable 80, and impeller 42, such that these components rotate together, such as when a motor is activated. An inflow region or an outflow region may include a blood mover. If blood mover 164 were disposed in the inflow, the axial orientation could be swapped, such that distal in FIG. 3 would be proximal and proximal in FIG. 3 would be distal.

FIG. 3 illustrates a merely exemplary embodiment of a blood mover, which again, may take the place of element 64 in FIGS. 1 and 2 (or which may be incorporated into the inflow region). As is set forth above, blood mover 164 need not be part of a bearing housing, although in the embodiment in FIGS. 1 and 2, it is a part of a bearing housing 60. Although the blood mover 164 is described as being adjacent the impeller, one will appreciate from the description herein that the blood mover may be positioned in a variety of other locations. Moreover, a plurality of blood movers may be provided. The blood movers may be configured and positioned to address a variety of locations and types of blood stagnation zones.

In the embodiments herein, blood mover 164 may be disposed within the outflow region of the blood pump, such as is shown in FIGS. 1 and 2. Alternatively or additionally, it may be disposed in the inflow region. Blood mover 164 includes body 168, which includes surface 169. Surface 169 in this example is optionally conical, with a radial dimension increasing in the proximal direction. In this embodiment, blood mover 164 includes a plurality of fins 166 radially extending from surface 169 (three in this example), although it is conceivable that the blood mover may only include a single fin. Blood movers may include from one to ten fins, or more. Fins 166 includes a fin height as shown in FIG. 3 and a fin length as shown in FIG. 3. While the heights and lengths are shown as the same, in alternative embodiments any of the fin heights may be different from another fin height, and any of the fin lengths may be different than any of the fin lengths. The fins may have a greatest radial dimension that is the same as a greatest radial dimension of the body 168, as is shown generally in FIG. 3. In alternative embodiments the fins may extend further radially than a proximal end of the blood mover. The blood movers herein may include a proximal region (void of fins) that has a cylindrical configuration, an example of which is shown in FIG. 3. Blood mover 164 includes a central lumen 164 defined by an inner surface, wherein the inner surface may be attached directly to an impeller shaft, such as impeller shaft 90. The blood mover may alternatively be coupled directly to a drive cable.

The fins in this embodiment are axially extending, that is, they do not circumferentially wrap around surface 169. In alternative embodiments any of the fins may wrap circumferentially around surface 169 to at least some extent.

The blood movers herein are generally configured to at least one of reduce blood stagnation or recirculation compared to a blood pump without the blood mover. At least a portion of the blood movers herein may be disposed completely within the outflow region of the pump, such as in example in FIGS. 1 and 2. At least a portion of the blood movers herein may be disposed completely within the inflow region of the pump (not explicitly shown). Any of the blood pumps herein may include a blood mover in the inflow and the outflow.

The blood movers herein are generally configured to move blood. In the embodiments herein, the blood movers may be adapted to rotate, and may be configured to move blood when they are rotated. Blood mover 164 includes a plurality of radially extending fins 166, and when the blood mover is rotated, the fins and one or more surfaces cause blood in the vicinity of blood mover to move. By moving blood in the vicinity of the blood mover, the blood mover is configured to reduce recirculation or stagnation, which as set forth herein may reduce the likelihood of blood clots and thrombosis.

In the embodiments herein, element 64 may be part of a bearing housing that rotates when the drive shaft rotates. Configuring a rotating bearing housing element as a blood mover may thus take advantage of a rotating component by incorporating blood moving functionality into the rotating component.

In alternative embodiments, any of the systems herein may be configured to rotate the one or more blood movers at a different rotational speed than the impeller(s). For example, without limitation, the one or more blood movers may be coupled to a different driveshaft/drive cable than that of the one or more impellers. This type of arrangement may be used to enhance the effect of the blood mover.

In alternative embodiments, any of the systems herein may be configured to implement a passive blood mover or movers that is not coupled to any driveshaft. In these embodiments, the flow of blood (e.g., from the one or more impellers) may be configured to passively rotate the blood mover(s) at the inlet or outlet of the blood pump. The vanes or fins of the blood mover, as illustrated and described above, can be configured to catch primary blood flow as it's being pulled through the impellers. This can potentially disturb the flow of blood in a stagnation zone, such as stagnation within the inlet, outlet, or in between struts or arms of the inlet/outlet. In some examples, a portion of the passive blood mover(s) may push blood back in the opposite direction, or alternatively generate flow to assist the main flow from the impeller(s).

In any of the embodiments herein, the blood mover may comprise surface 169 and one or more fins 166 that together work to move blood when the blood mover is rotated. The radially projecting fins by themselves may also, individually or together, be considered to be blood movers herein when rotated.

When rotated, the fins 166 and surface 169 may cause blood to move and perform one or more of the following functions: reduce blood recirculation or stagnation. As set forth above, moving blood in this capacity may reduce the likelihood of blood clotting, thereby reducing the likelihood of thrombosis.

The blood movers herein may be positioned within the outflow region of the pump, such as is shown in the example of FIGS. 1 and 2. The outflow region generally refers to an axial region between a blood conduit and proximal ends of struts. The blood conduit proximal end may be curved as shown in FIGS. 1 and 2 (rather than having a circular section at its proximal end), so the outflow region may have a distal end that is similarly considered to be curvilinear.

In the embodiments herein, the blood mover has a radial dimension that is small enough to avoid having to be a collapsible and expandable structure, and yet can still move blood in its vicinity when rotated. The blood movers herein may thus be relatively stiffer than a collapsible and expandable impeller (and may be significantly stiffer), which may need to be flexible enough to collapse for delivery and removal from the patient. In some examples, the blood movers may be made of rigid materials such as a metallic material. The blood movers herein may have a radially outermost dimension that is less than an impeller outermost dimension (as shown in FIGS. 1 and 2), and may be less than the impeller radial dimension when the impeller is collapsed as well as when the impeller is expanded.

The blood movers herein may additionally or alternatively be disposed in a pump inflow, which may provide any of the functions herein for the inflow region. In some embodiments, the blood mover may have surface 169, which decreases in dimension in the proximal direction (if the blood mover from FIG. 3 were rotated 180 degrees).

In alternative embodiments, the one or more blood movers may be sized and configured to be collapsible and expandable. For example, any of the fins may be adapted (including made of a material) and dimensioned to be collapsible (e.g., similar to impeller blades) for delivery and sheathing, yet expandable upon deployment from a sheath.

1. A intravascular blood pump, comprising:
a collapsible pump portion;
an impeller disposed within the collapsible pump portion near a pump outflow; and
a rotatable blood mover axially spaced from the impeller and being disposed at least partially within the pump outflow.

2. The blood pump of claim 1, wherein the rotatable blood mover is rotationally coupled to the impeller.

3. The blood pump of claim 1, wherein the rotatable blood mover is adapted to be collapsible and expandable.

4. The blood pump of claim 1, wherein the rotatable blood mover is not adapted to be collapsible and expandable.

5. The blood pump of claim 1, wherein the rotatable blood mover is more rigid than the impeller.

6. The pump portion of any of claim 1, wherein the rotatable blood mover comprises a plurality of radially extending fins, and optionally includes a conical surface from which the plurality of fins extend.

7. The pump portion of claim 1, wherein the rotatable blood mover has a radially outermost dimension less than a radially outermost dimension of the impeller when expanded, both relative to a long axis of a drive cable, optionally wherein the radially outermost dimension of the blood mover is less than half of the radially outermost dimension of the impeller when expanded.

8. The blood pump of claim 1, wherein the rotatable blood mover is adapted to reduce at least one of blood recirculation or stagnation in the vicinity of the blood mover or the pump outflow.

9. The blood pump of claim 1, wherein the impeller is fully disposed in the pump outflow.

10. The blood pump of any of claim 1, wherein the pump outflow includes a radial flow component.

11. The blood pump of claim 1, wherein the blood mover is not in contact with the impeller.

12. The blood pump of claim 2, wherein the rotatable blood mover is rotationally coupled to the impeller with a drive shaft.

13. The blood pump of claim 12, wherein the rotatable blood mover is part of a bearing housing that rotates when the drive shaft rotates.

14. A intravascular blood pump, comprising:
a collapsible pump portion;
an impeller disposed within the collapsible pump portion near a pump inflow; and
a rotatable blood mover axially spaced from the impeller and being disposed at least partially within the pump inflow.

15. The blood pump of claim 14, wherein the rotatable blood mover is rotationally coupled to the impeller.

16. The blood pump of claim 14, wherein the rotatable blood mover is adapted to be collapsible and expandable

17. The blood pump of claim 14, wherein the rotatable blood mover is not adapted to be collapsible and expandable.

18. The blood pump of claim 14, wherein the rotatable blood mover is more rigid than the impeller.

19. The blood pump of any of claim 14, wherein the rotatable blood mover comprises a plurality of radially extending fins, and optionally includes a conical surface from which the plurality of fins extend.

20. The blood pump of claim 14, wherein the rotatable blood mover has a radially outermost dimension less than a radially outermost dimension of the impeller when expanded, both relative to a long axis of a drive cable, optionally wherein the radially outermost dimension of the blood mover is less than half of the radially outermost dimension of the impeller when expanded.

21-39. (canceled)
